(19) United States
(12) Patent Application Publication
(10) Pub. No.: US 2007/0015763 A1
(43) Pub. Date: Jan. 18, 2007

(54) Treatment of psychosis associated with parkinson's disease and subcortical dementias using a combination of an atypical antipsychotic with a dopamine agonist

(75) Inventor: Steve Romano, New York, NY (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/179,390

(22) Filed: Jul. 12, 2005

(51) Int. Cl. A61K 31/519; A61K 31/498; A61K 31/48; A61K 31/198; A61K 31/137

(52) U.S. Cl. 514/250; 514/259.41; 514/284; 514/649; 514/567

(57) ABSTRACT

This invention relates to combinations of an atypical antipsychotic, for example ziprasidone, and a dopamine agonist, kits containing such combinations, pharmaceutical compositions comprising such combinations, and methods of using such combinations to treat patients suffering from psychosis and movement disorders associated with Parkinson's disease and subcortical dementias.

### FIELD OF THE INVENTION

The present invention relates to pharmaceutical combinations comprising ziprasidone. The invention also pertains to pharmaceutical combinations comprising atypical antipsychotics. The invention also relates to methods to treat patients, including humans, suffering from psychosis associated with Parkinson's Disease or other subcortical dementias.

### BACKGROUND OF THE INVENTION

Parkinson's disease is among the most common movement disorders in the United States. Parkinsonism refers to disturbances of motor function and movements that are characteristic of Parkinson's disease. These disturbances are usually thought to be caused by the loss of dopaminergic projections from the substantia nigra pars compacta in the midbrain to the corpus striatum. Thus, symptoms such as tremor, rigidity (often with ratchetlike “cogwheeling bradykinesia” (slow movements), akinesia (slow onset of movements), and a stooped posture with slow strides and a shuffling gait may respond to a number of medications that work by enhancing or replacing dopaminergic activity in the corpus striatum.

For many years, the gold standard for treatment of Parkinson's disease has been levodopa combined with the peripheral decarboxylase inhibitor carbidopa. Although the half-life of carbidopa-levodopa in the blood is only about two hours, a sustained functional response can usually be achieved in patients with mild disease after the combination is administered only twice or three times daily. This effect is thought to occur from the uptake of excessive levodopa in the stratum of the brain by high affinity sites on dopaminergic nerve endings. The levodopa is then converted to dopamine and released slowly. With progression of the focal disease and loss of these high-affinity uptake sites, postsynaptic levels of levodopa and dopamine follow blood levels more closely resulting in rapidly shifting or unpredictable patient response.

One approach commonly used to treat worsening Parkinson's disease is the use of long-acting dopamine agonists, such as pergolide, pramipexole, or ropinirole; the monoamine oxidase (MAO-B) inhibitor selegiline; amantadine; or possibly an anticholinergic agent. In some patients catechol-ortho-methyltransferase (COMT) inhibitors such as entacapone or tolcapone may be added to prolong the half-life of levodopa. In some patients these medications can be used singly or in combination for as long as several years before the addition of carbidopa-levodopa is required to achieve the best clinical response.

Of major concern are the CNS effects that may occur with the dopamine agonists. These include sedation and confusion, but, more notably, hallucinations. Psychotic symptoms are aggravated by dopamine-related medications used to treat the Parkinson's disease and frequently occur as adverse effects of those medications in patients. Behavioral disturbances caused by dopamine-related medications can occur not only in patients with Parkinsonism associated with loss of dopaminergic neurons but also in Parkinsonian patients without a dopamine deficiency. That is, in advanced cases of Parkinsonism, dementia may be present.

As Parkinsonism becomes more widespread cognitive and behavioral symptoms often become more prominent and can include hallucinations, delusions, episodic confusion, and frontal lobe dysfunction, even before the emergence of overt dementia.

The psychotic symptoms that occur commonly in Parkinson's patients who have dementia may reflect extranigral pathological changes. Persistent threatening hallucinations or unusual or paranoid delusions are often extremely disturbing not only to the patient but to his or her family and the health care professionals involved in the patient's care. Such symptoms may cause such patients to be hospitalized, institutionalized, or placed into a nursing home.

Additionally, patients with Parkinson's disease can also have concurrent conditions, such as Alzheimer's disease or multiple infarcts, that contribute to clinical psychotic symptoms.

Subcortical dementias result from dysfunction in the parts of the brain that are beneath the cortex. Subcortical dementias are associated with motor impairment and involuntary movements. Depending on the type of subcortical dementia the movement disorders may include Parkinsonism with prominent gait disturbances in conjunction with pyramidal tract signs. Features characteristic of subcortical dementia include: memory impairments (anterograde and retrograde), bradyphrenia, difficulties on tasks with a visuospatial component (picture arrangement; block design; object assembly), frontal lobe symptoms (difficulties planning, using feedback, generating behaviors), personality and mood changes, and movement disorders. Patients with subcortical dementia often exhibit troublesome and disruptive behaviors and symptoms of psychosis in addition to the cognitive impairments. Pharmacological management of subcortical dementias include atypical antipsychotics. Therefore, patients with subcortical dementias may have both symptoms of psychosis together with movement disorders.

Subcortical dementias include a variety of syndromes: Parkinson's disease with dementia; multiple system atrophy; progressive supranuclear palsy; dementia due to Huntington's disease (see DSM-IV-TR, Amer Psychiat Assoc, 2000, p. 165); dementia due to HIV disease (see DSM-IV-TR, Amer Psychiat Assoc, 2000, p. 165); and dementia due to Creutzfeldt-Jakob disease. For example, in Huntington's disease, 90% of patients may have dementia by a mean age of 48.3 years. HIV related neuropathology is characterized by subcortical dementia, which develops in approximately 60% of patients, together with motor difficulties, and affective disorders (Navia B. A. et al., 19 Ann. Neurol. 6, 198; Bridge, AIDS AND HIV CNS DISEASE: A NEUROPSYCHIATRIC DISORDER, Nat'l Inst. Mental Health, NIH, 1988).

Mental illness is particularly difficult to treat in that not all patients react similarly to the same treatment regimen. Patients often require multiple drug therapies. There also exists a large number of untreated individuals and treatment-resistant patients in need of effective therapy.

Exacerbating this is the problem of patient noncompliance. For example, it is conventionally thought that substantial numbers of patients with mental illnesses are not compliant or only partially compliant with their medication. Poor compliance can cause relapses thereby negating whatever benefits were achieved through treatment in the first place.

Simplification of the regimen by combining several therapeutic agents, reduces the opportunity for patient noncompliance as occurs with a more rigorous schedule. Accordingly, there is a need for new techniques efficacious for the treatment of, e.g. psychosis associated with Parkinson's disease or subcortical dementias.

There is also a need in the art for new and improved treatments for other diseases, disorders, and syndromes that are characterized by symptoms of psychosis associated with Parkinson's disease or subcortical dementias and/or psychotic disorders or conditions.

The present invention is directed to compositions, which reduce or overcome these disadvantages. The invention, for the first time, provides pharmaceutical combinations of ziprasidone and dopamine agonists for the treatment of psychosis associated with Parkinson's disease and subcortical dementias.

### SUMMARY OF THE INVENTION

The present invention is directed to pharmaceutical compositions, therapeutic methods of treatment, and kits which employ an atypical antipsychotic together with a dopamine agonist.

According to the invention, it has surprisingly been found that the pharmaceutical combinations of the present invention can provide synergistic and additive effects with symptomatic relief of both the psychosis and movement disorders associated with Parkinson's disease or subcortical dementias, less side effects, a reduction in use of concomitant psychotropic medications such as antidepressants, sedatives and mood stabilizers such as lithium, and prevention of future decline.

Thus, in one aspect, the present invention provides a combination of an atypical antipsychotic agent and a dopamine agonist. Atypical antipsychotics which can be used in the present invention include olanazapine, clozapine, risperidone, sertindole, quetiapine, aripiprazole, amisuipride and ziprasidone. In general, pharmaceutical combinations and methods of treatment using ziprasidone as the first therapeutic agent are preferred.

A further feature of the present invention is a method of reducing the amount of the atypical antipsychotic agent required to produce an antipsychotic effect which comprises treating a patient with a therapeutically effective amount of a drug combination according to the present invention.

It is also a feature of this invention that the use of such drug combinations will enhance the effect of the atypical antipsychotic agent to be used and therefore allow reduced quantities of the antipsychotic agent to be employed and, therefore allow better management of drug-related toxicity and side effects.

The invention offers advantages over previous methods for treating neuropsychiatric disorders in affected patients. The method of the present invention will enhance the effect of the dopamine agonist used and therefore allow reduced quantities of the dopamine agonist to be used and, therefore allow better management of drug-related toxicity and side effects.

The invention offers advantages over previous methods for treating neuropsychiatric disorders. For example, in the method of treatment of the present invention, the atypical antipsychotic counteracts the nausea and vomiting and stupor effects of individual dopamine agonists.

A further advantage of the present invention is simplification of the regimen by combining an atypical antipsychotic and a dopamine agonist in a single dosage form or kit, thereby reducing the opportunity for patient noncompliance as occurs with a more rigorous schedule.

Other features and advantages of the invention will be apparent from the following detailed description and from the claims.

### DETAILED DESCRIPTION OF THE INVENTION

The present invention is directed to pharmaceutical compositions comprising an amount of an atypical antipsychotic, for example ziprasidone or a prodrug thereof, or a pharmaceutically acceptable salt of ziprasidone or said prodrug; and an amount of a dopamine agonist, and, a pharmaceutically acceptable vehicle, carrier or diluent, wherein the amounts of the ziprasidone, ziprasidone prodrug, ziprasidone salt, or ziprasidone prodrug salt and the dopamine agonist are together therapeutically effective.

The present invention is also directed to a therapeutic method and pharmaceutical compositions comprising an atypical antipsychotic, for example ziprasidone, and a dopamine agonist useful for treating psychosis and movement disorders associated with Parkinson's disease or subcortical dementias.

This invention is also directed to kits for achieving a therapeutic effect in a patient which include an amount of an atypical antipsychotic, for example ziprasidone or a prodrug thereof, or a pharmaceutically acceptable salt of ziprasidone or said prodrug, and a pharmaceutically acceptable vehicle, carrier or diluent in a first unit dosage form; and an amount of a dopamine agonist, and a pharmaceutically acceptable vehicle, carrier or diluent in a second unit dosage form, and a container, wherein the amounts of the atypical antipsychotic and the dopamine agonist are together therapeutically effective.

This invention is also directed to methods of treating a patient in need of therapy by administering to the patient an amount of an atypical antipsychotic, for example ziprasidone or a prodrug thereof, or a pharmaceutically acceptable salt of ziprasidone, or said prodrug, and an amount of a dopamine agonist wherein the amounts of the atypical antipsychotic and the dopamine agonist are together therapeutically effective.

This invention is also directed to methods for treating a patient in need of therapy by administering to the patient a pharmaceutical composition comprising

a) an amount of a first compound, the first compound being an atypical antipsychotic, for example ziprasidone or a pharmaceutically acceptable salt of ziprasidone, a prodrug of ziprasidone, or a pharmaceutically acceptable salt of a ziprasidone prodrug; and

b) an amount of a second compound, the second compound being a dopamine agonist; and,

a pharmaceutically acceptable vehicle, carrier or diluent.

wherein the amounts of the atypical antipsychotic and the dopamine agonist are together therapeutically effective.

The methods of this invention provide therapeutic treatment of both the psychosis and movement disorders associated with Parkinson's disease and other subcortical dementias.

“Psychosis associated with Parkinson's disease or other subcortical dementias” and like phrases refer to symptoms of psychosis such as delusions, hallucinations and paranoias, either as a pathophysiological consequence of Parkinson's disease or a subcortical dementia or with co-existing neuropathology indicative of Parkinson's disease or a subcortical dementia.

“Movement disorder associated with Parkinson's disease or other subcortical dementias” and like phrases refer to dysarthria or impairments of voluntary movement either as a pathophysiological consequence of Parkinson's disease or a subcortical dementia or with co-existing neuropathology indicative of Parkinson's disease or a subcortical dementia.

“Subcortical dementias” refers to dysfunction in the parts of the brain that are beneath the cortex. Subcortical dementias include mixed cortical-subcortical dementias such as corticobasal ganglionic degeneration and diffuse Lewy body disease (Lewy body dementia). Psychosis associated with Parkinson's disease or subcortical dementias which may be treated by the methods of this invention include, inter alia, psychosis associated with the following disorders and conditions: Parkinson's disease; subcortical dementia due to other general medical condition, including but not limited to, Human Immunodeficiency Virus, Parkinson's disease, vascular dementia, Huntington's disease, multiple sclerosis, multiple system atrophy; progressive supranuclear palsy; Creutzfeldt-Jakob disease; corticodentatonigral degeneration; Gerstmann-Straussler Scheincker disease; Familial Olivoponto cerebellar degeneration; Familial myoclonic dementia; Amyotropic Lateral Sclerosis with Parkinsonism or Dementia; Dementia and Parkinsonism with Non-Alzheimer Amylooid Plaques; thalamic dementia; and familial non-alzheimer dementia. Other psychoses associated with Parkinson's disease or subcortical dementias which may be treated by the methods of this invention appear in DSM-IV, 4^{th} ed., pp. 135-171 and 297-343, which is incorporated herein by reference.

“Dementia” refers to global deterioration of intellectual functioning in clear consciousness, and is characterized by one or more symptoms of disorientation, impaired memory, impaired judgment, and/or impaired intellect. The symptoms of “dementia” are generally worse than, and can encompass, the symptoms of “psychosis associated with Parkinson's disease or subcortical dementias.”

The term “psychosis”, “psychotic disorder”, and other such terms, as used herein, unless otherwise specified refer to a disorder or condition comprising one or more psychotic symptoms. Psychotic symptoms are known to those of ordinary skill in the art. Psychotic symptoms include, for example, delusions, prominent hallucinations, disorganized speech, and disorganized or catatonic behavior.

The terms “patient”, “subject”, and other such terms, as used herein, unless otherwise indicated, include all mammals, including, for example, dogs, cats and humans. Preferably, “patient” or “subject” refers to a human.

In the methods described herein, the dopamine agonists and atypical antipsychotics of the invention alleviate (e.g., reduce or eliminate) at least one symptom, preferably two, three, or all symptoms of the disease, disorder or syndrome being treated. Preferably, the dopamine agonists and atypical antipsychotics alleviate the symptoms of psychosis and movement disorders associated with Parkinson's disease or a subcortical dementia.

Preferred dopamine agonists for use in the combinations, pharmaceutical compositions, methods and kits of this invention include, but are not limited to, levodopa, bromocriptine, carbidopa (Lodosyn®), carbidopa/levodopa (Sinemet®) pramipexole (Mirapex®), pergolide mesylate (Permax®), and ropinirole (Requip®), including all prodrugs thereof, pharmaceutically acceptable salts thereof, and pharmaceutically acceptable salts of such prodrugs.

More preferred dopamine agonists for use in the combinations, pharmaceutical compositions, methods and kits of this invention include levodopa/carbidopa (Sinemet®) and levodopa, including all prodrugs thereof, and pharmaceutically acceptable salts thereof, and pharmaceutically acceptable salts of such prodrugs.

A particularly preferred dopamine agonist for use in the combinations, pharmaceutical compositions, methods and kits of this invention is levodopa, including all prodrugs thereof, and pharmaceutically acceptable salts thereof, and pharmaceutically acceptable salts of such prodrugs.

Another particularly preferred dopamine agonist for use in the combinations, pharmaceutical compositions, methods and kits of this invention is levodopa/carbidopa, including all prodrugs thereof, and pharmaceutically acceptable salts thereof, and pharmaceutically acceptable salts of such prodrugs.

The combinations of this invention comprise at least two active components: ziprasidone or a prodrug thereof, or a pharmaceutically acceptable salt of ziprasidone or said prodrug; and a dopamine agonist. The compositions of this invention include a pharmaceutically acceptable vehicle, carrier or diluent.

The combinations may result in synergistic action allowing a lower dose of the atypical antipsychotic to be administered while achieving at least the same or an improved psychotropic effect compared to unitary administration of the atypical antipsychotic. The dosage of the atypical antipsychotic may be reduced by about 25-90%, for example, about 40-80%, and typically about 50-70%. The reduction in amount of antipsychotic required may depend on the amount of the second therapeutic agent given.

Another advantage of the combination is that the synergistic action allows the dose of the dopamine agonist to be decreased thereby resulting in fewer side effects. For example, levodopa causes dose-dependent nausea and vomiting. A combination which contains decreased dosages of levodopa may result in less nausea and vomiting for the patient.

The selection of the dosage of the first therapeutic agent (i.e., an atypical antipsychotic) and second therapeutic agent (i.e., a dopamine agonist) is that which can provide relief to the patient as measured by a reduction or amelioration of symptoms associated with the disorder or condition of the patient. The dosage of each component depends on several factors such as the potency of the selected specific compound, the mode of administration, the age and weight of the patient, the severity of the condition to be treated, and the like. Adjustment of the dosages is considered to be within the skill of the artisan. To the extent necessary for completeness, the synthesis of the components of the compositions and dosages are as described in the patents listed herein or the Physicians' Desk Reference, 57th ed., Thompson, 2003, which are expressly incorporated by reference. Desirably, when ziprasidone is selected as the active agent, the daily dose contains from about 5 mg to about 460 mg.

More preferably, each dose of the first component (i.e., ziprasidone) contains about 10 mg to about 200 mg of the ziprasidone, and even more preferably, each dose contains from about 20 mg to about 160 mg of ziprasidone. Pediatric dosages may be less such as, for example, in the range of about 0.5 mg to about 40 mg daily. The full daily dosage may be administered in one or two doses, for example.

General outlines of the dosages for the atypical antipsychotics and dopamine agonists, and some preferred dosages, are provided herein. This list is not intended to be complete but is merely a guideline for any of the desired combinations of the present invention:

Olanzapine: from about 0.25 to about 100 mg, once/day; preferably, from about 1 to about 30 mg, once/day; and most preferably about 1 to about 25 mg once/day;

Clozapine: from about 12.5 to about 900 mg daily; preferably, from about 150 to about 450 mg daily;

Risperidone: from about 0.25 to about 16 mg daily; preferably from about 2-8 mg daily;

Sertindole: from about 0.0001 to about 1.0 mg/kg daily;

Quetiapine: from about 1.0 to about 40 mg/kg given once daily or in divided doses;

Asenapine: from about 0.005 to about 60 mg total per day, given as a single dose or in divided doses.

The Table below provides additional dosage ranges:

Drug Name

| Brand | | |
|---|---|---|
| name | Generic Name | Dosage Range |
| Permax | Pergolide Mesylate | From about: 0.05 mg |
| | | To about: 5 mg |
| Lodosyn | Carbidopa | From about: 25 mg. |
| | | To about: 300 mg |
| Mirapex | Pramipexole | From about: 0.125 mg |
| | Dihydrochloride | To about: 6 mg. |
| Requip | Ropinirole | From about: 0.25 mg |
| | Hydrochloride | To about: 24 mg |
| Sinemet | Carbidopa/levodopa | From about: 25 mg/100 mg |
| | | To about: 300 mg/2400 mg |
| Various | Levodopa | From about: 100 mg |
| | | To about: 2400 mg |
| Various | Bromocriptine | From about: 1.25 mg |
| | | To about: 100 mg |

It is to be understood that other dopamine agonists and/or atypical antipsychotics can be used in the present invention.

In general, in accordance with this invention, a combination containing an atypical antipsychotic in combination with two or more dopamine agonists can be used. As an example, ziprasidone can be combined with levodopa/carbidopa and further combined with entacapone.

The presently preferred atypical antipsychotic to use in the invention is ziprasidone. Ziprasidone (5-[2-[4-(1,2-benzisothiazol-3-yl)piperazin-1-yl]ethyl]-6-chloroindolin-2-one) is a benzisothiazolyl piperazine-type atypical antipsychotic with in vitro activity as a 5-HT_{1A} receptor agonist and an inhibitor of serotonin and norepinephrine reuptake (U.S. Pat. No. 4,831,031).

Ziprasidone is efficacious for the treatment of patients with schizophrenia and schizomood disorders, refractory schizophrenia, psychosis associated with Parkinson's disease or subcortical dementias in schizophrenia, affective and anxiety symptoms associated with schizoaffective disorder and bipolar disorder. The drug is considered a safe and efficacious atypical antipsychotic (Charles Caley & Chandra Cooper, 36 Ann. Pharmacother. 839-51, 2002).

The use of ziprasidone in psychosis is described in, e.g., in U.S. Pat. Nos. 6,245,766; 6,245,765; 6,387,904; 5,312,925; 4,831,031; and European EP 0901789, published Mar. 17, 1999, all of which are incorporated herein by reference.

Other atypical antipsychotics which can be employed include, but are not limited to: Olanzapine, 2-methyl-4-(4-methyl-1-piperazinyl)-10H-thieno[2,3-b][1,5]benzodiazepine. Olanzapine is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated by reference in its entirety; Clozapine, 8-chloro-11-(4-methyl-1-piperazinyl)-5H-dibenzo[b,e][1,4]diazepine. Clozapine is described in U.S. Pat. No. 3,539,573, which is herein incorporated by reference in its entirety. Clinical efficacy in the treatment of schizophrenia is described (Hanes, et al., 24 Psychopharmacol. Bull. 62, 1988);

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663, which is herein incorporated by reference in its entirety;

Sertindole, 1-[2-[4-[5-chloro-1-(4-fluorophenyl)-1H-indol-3-yl]-1-piperidinyl ]ethyl]imidazolidin-2-one. Sertindole is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entirety;

Quetiapine, 5-[2-(4-dibenzo[b,f][1,4]thiazepin-11-yl-1-piperazinyl)ethoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt; Aripiprazole, 7-{4-[4-(2,3-dichlorophenyl)-1-piperazinyl]-butoxy}-3-,4-dihydro carbostryril or 7-{4-[4-(2,3-dichlorophenyl)-1-piperazinyl]-butoxy}-3,4-dihydro-2(1H)-quinolino, is an atypical antipsychotic agent used for the treatment of schizophrenia and described in U.S. Pat. No. 4,734,416 and U.S. Pat. No. 5,006,528, which are herein incorporated by reference in their entirety; and Amisulpride. Amisulpride is described in U.S. Pat. No. 4,401,822.

Asenapine, trans-5-chloro-2-methyl-2,3,3a, 12b-tetrahydro-1H-dibenz [2,3:6,7]oxepino[4,5-c]pyrrole. Preparation and use of asenapine is described in U.S. Pat. Nos. 4,145,434 and 5,763,476, which are incorporated herein in their entireties by reference.

A preferred combination is ziprasidone with a dopamine agonist. The term “dopamine agonist”, where used in the description and the appended claims, is synonymous with the term “dopaminergic agent”, These terms are used interchangeably throughout the description and appended claims.

The term “dopamine agonist” as used throughout, refers to compounds which mimic the action of dopamine. The meaning of the term is known in the art.

Besides the dopaminergic agents described above, examples of other suitable dopamine agonists that can be employed in the methods and pharmaceutical compositions of this invention, as described above, are found in commonly assigned U.S. patent application Ser. No. 2003/0045449, filed May 1, 2002, and include, ropinole, L-dopa in combination with an L-dopa decarboxylase inhibitor such as carbidopa or benserazide, bromocriptine, dihydroergocryptine, etisulergine, AF-14, alaptide, pergolide, piribedil, dopamine D1 receptor agonists such as A-68939, A-77636, dihydrexine, and SKF-38393; dopamine D2 receptor agonists such as carbergoline, lisuride, N-0434, naxagolide, PD-118440, pramipexole, quinpirole and ropinirole; dopamine/beta.-adrenergic receptor agonists such as DPDMS and dopexamine; dopamine/5-HT uptake inhibitor/5-HT-1A agonists such as roxindole; dopamine/opiate receptor agonists such as NIH-10494; α-2-adrenergic antagonist/idopamine agonists such as terguride; α-2-adrenergic antagonist/dopamine D2 agonists such as ergolines and talipexole; dopamine uptake inhibitors such as GBR-12909, GBR-13069, GYKI-52895, and NS-2141; monoamine oxidase-B inhibitors such as selegiline, N-(2-butyl)-N-methylpropargylamine, N-methyl-N-(2-pentyl)propargylamine, AGN-1133, ergot derivatives, lazabemide, LU-53439, MD-280040 and mofegiline; and COMT inhibitors such as CGP-28014.

The above dopamine agonists are not all-inclusive. It is to be understood that other dopamine agonists can be used in the present invention. The dopamine agonists disclosed herein are prepared by methods well known to those skilled in the art. Specifically, the following patents, patent applications, and references, each of which is incorporated herein by reference, exemplify dopamine agonists which can be used in the combinations, pharmaceutical compositions, methods and kits of this invention, and refer to methods of preparing those dopamine agonists: U.S. Pat. No. 3,253,023 (specifically, levodopa); U.S. Pat. No. 3,462,536 and Sletzinger et al., 6 J. Med. Chem. 101 (1963) (specifically, carbidopa); U.S. Pat. No. 4,166,182 (specifically, pergolide); U.S. Pat. No. 4,886,812 and Schneider, C. S., 30 J. Med. Chem. 494 (1987) (specifically, pramipexole); U.S. Pat. No. 5,837,724 (specifically, ropinirole); and U.S. Pat. No. 3,752,814 and 3,752,888 (specifically, bromocriptine).

Carbidopa, (-)-L-α-hydrazino-α-methyl-β-(3,4-dihydroxybenzene) propionic acid monohydrate, is a peripheral inhibitor of dopa decarboxylase indicated as an adjunct to levodopa for the treatment of symptoms of idiopathic Parkinson's disease, postencephalitic parkinsonism, and symptomatic Parkinsonism which may follow injury by carbon monoxide and/or manganese intoxication. Carbidopa and its methods of use is described in U.S. Pat. Nos. 3,462,536 incorporated herein by reference in its entirety.

Levodopa, (-)-L-α-amino-β-(3,4-dihydrobenzene) propionic acid, is a dopamine agonist useful for the treatment Parkinson's disease. Levodopa and its methods of use are as described in the Physician's Desk Reference, 57^{th} ed., 2003, p. 1110-1111, which is incorporated herein by reference in its entirety.

For use in medicine, pharmaceutically acceptable salts can be useful in the preparation of the compounds according to the invention. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

Where the dopamine agonists employed in the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of dopamine agonists or ziprasidone containing free carboxylic acids can be readily prepared by reacting the free acid form of the dopamine agonist with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of dopamine agonists or ziprasidone containing free amine groups can be readily prepared by reacting the free base form of the dopamine agonist with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

For the purposes of this specification, psychosis and dementias associated with Parkinson's disease, and subcortical dementias, such as due to medical conditions (e.g., Huntington's disease, dementia due to HIV infection), are as defined in accordance with the DSM-IV criteria.

The efficacy of the methods and compositions of the present invention in the treatment of subcortical dementias can be evaluated in human clinical trials using scales such as Neuropsychiatric Inventory Score to evaluate the efficacy of compounds of the present invention. Additionally, the state of the disease before and after treatment may be assessed by various commonly accepted mental-state examinations, including the information- concentration-orientation test (Blessed, 12 Br. J. Psychiatr. Res. 189-198, 1968), the Mini Mental State Examination (MMSE) (Folstein et al., 12 J. Psychiatr. Res. 189-195, 1975) and the Global Deterioration Scale (Reisberg, 140 Am. J. Psychiatry, 734-739, 1983). The aforementioned references describing these assays are incorporated herein by reference in their entireties.

Psychotic disorders or conditions, such as schizophrenia, schizoaffective disorder, schizophreniform disorder, and schizotypical disorder are conditions in which neuroleptic therapy, such as treatment with atypical antipsychotics, is important. According to the present invention, these conditions can now also be treated with an atypical antipsychotic in combination with, for example, levodopa/carbidopa.

The atypical antipsychotics can be administered simultaneously with the dopamine agonists, either as separate dosage forms for example in a kit product, or as one combined dosage form containing both the atypical antipsychotic and the dopamine agonist.

The effects of a pharmaceutical composition containing ziprasidone and a dopamine agonist of the present invention can be examined by using one or more of the published models of neuropsychiatric evaluation well known in the art. The effects of a pharmaceutical composition containing ziprasidone and a dopamine agonist of the present invention can also be examined by using one or more of the published symptom scoring models of Parkinson's disease well known in the art.

The combinations comprising ziprasidone and a dopamine agonist of the present invention are particularly useful for the prevention of, reducing the development of, or reversal of, psychosis associated with Parkinson's disease or a subcortical dementia and are, therefore, particularly useful in the treatment of Parkinson's disease and other dementias.

The pharmaceutical compositions containing ziprasidone and a dopamine agonist of the present invention are particularly useful for the prevention of, reducing the development of, or reversal of, psychotic disorders, conditions or symptoms and are, therefore, particularly useful in the treatment of schizophrenia, schizophreniform disorder, schizoaffective disorder or delusional disorder. This can be demonstrated, for example, by measuring markers such Positive or Negative Syndrome Scale (PANSS) and Scales for the Assessment of Negative Symptoms (SANS) or BPRS scores (Kay et al, Schizophrenia Bulletin 13:261-276, 1987), which reference is incorporated in its entirety by reference herein, or in various animal models such as the PCP or methamphetamine induced locomotor test, or the conditioned avoidance response test.

In general, ziprasidone employed in the combinations, pharmaceutical compositions, methods and kits of this invention, will be administered at dosages between about 5 and about 460 mg per day, preferably from about 10 mg to about 200 mg, more preferably 20 mg to 160 mg per day, and most preferably from about 20 mg to 80 mg per day, together with therapeutically effective amounts of the second therapeutic agent in single or divided doses.

The term “therapeutically effective amount” as used herein refers to a sufficient amount of the combination to treat psychosis associated with Parkinson's disease or subcortical dementias and psychotic disorders or conditions at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; and the age of the patient. However, some variation in dosage will necessarily occur depending upon the condition of the subject being treated. The person responsible for administration will, in any event, determine the appropriate dose for the individual subject.

The following dosage amounts and other dosage amounts set forth elsewhere in this description and in the appended claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and in the appended claims, are daily doses.

In preferred embodiments, the above dopamine agonists used in the combinations, pharmaceutical compositions, methods and kits of this invention will be administered to treat the conditions described herein in doses of about 0.1 milligram to about 1000 milligrams per day, preferably about 1 milligram to about 500 milligrams per day, more preferably in a dosage amount of about 2 milligrams to about 100 milligrams per day in single or divided doses. However, some variation in dosage will necessarily occur depending upon the condition, age as well as factors which may alter pharmacokinetics of absorption, distribution, metabolism and excretion in the subject being treated. The person responsible for administration will, in any event, determine the appropriate dose for the individual subject.

In preferred embodiments of the methods described herein, when used as the dopamine agonist in this invention, carbidopa/levodopa will be dosed at about 10/40mg (i.e., 10 mg carbidopa and 40 mg levodopa) to about 100/400mg (i.e., 100 mg carbidopa and 400 mg levodopa) per day; and preferably about 25/100mg (i.e., 25 mg carbidopa and 100 mg levodopa) to about 50/200mg (i.e., 50 mg carbidopa and 200 mg levodopa) per day; pergolide mesylate will be dosed at about 0.025 mg to about 5 mg per day, and preferably about 0.05 mg to about 3 mg per day.

One skilled in the art will appreciate that when the dopamine agonists are administered to children, the dose may be smaller than the dose that is administered to adults. The exact formulation, route of administration, and dosage can be chosen by the individual physician in view of the patient's condition. Dosage amount and interval can be adjusted individually to provide plasma levels of the active moiety which are sufficient to maintain therapeutic effects.

It will be recognized by a skilled person that the free base form or other salt forms of the above dopamine agonists can be used in this invention. Calculation of the dosage amount for these other forms of the free base form or other salt forms of a particular dopamine agonist is easily accomplished by performing a simple ratio relative to the molecular weights of the species involved.

The products of the present invention are of use in the treatment and/or prevention of a variety of disorders of the central nervous system. Such disorders include psychosis associated with Parkinson's disease or subcortical dementias disorders, subcortical dementias caused by traumatic brain injury, dementia due to other general medical conditions (e.g., human immunodeficiency virus disease, head trauma, Parkinson's disease, Huntington's disease), substance-induced persisting dementia (i.e., due to a drug of abuse, a medication, or toxin exposure), dementia due to multiple etiologies, or dementia not otherwise specified.

The products of the present invention have the advantage that they surprisingly provide greater relief from psychosis associated with Parkinson's disease or subcortical dementias and more rapid relief than would be expected from administration of either compound alone. The products of the present invention are also useful in reducing the complications associated with psychosis associated with Parkinson's disease or subcortical dementias.

The term “treating” as used herein, refers to reversing, alleviating, inhibiting the progress of, or preventing the disorder or condition to which such term applies, or one or more symptoms of such disorder or condition. The term “treatment”, as used herein, refers to the act of “treating” as defined immediately above.

For example, “treating a psychotic disorder” as used herein also encompasses treating one or more symptoms (positive, negative, and other associated features) of said disorder, for example, treating, delusions and/or hallucination associated therewith. Other examples of symptoms of psychotic disorders include disorganized speech, affective flattening, alogia, anhedonia, inappropriate affect, dysphoric mood (in the form of, for example, depression, anxiety or anger), and some indications of cognitive dysfunction.

In another embodiment, the compounds used in the present invention are useful to treat other disorders that may present with psychotic symptoms such as dementia of the Alzheimer's type; substance-induced delirium; and major depressive disorder with psychotic features.

The expression “prodrug” refers to compounds that are drug precursors which, following administration, release the drug in vivo via a chemical or physiological process (e.g., a prodrug on being brought to the physiological pH or through enzyme action is converted to the desired drug form).

The present invention includes within its scope the use of prodrugs of ziprasidone or prodrugs of dopamine agonists. In general, such prodrugs will be functional derivatives of these compounds which are readily convertible in vivo. Conventional procedures for the selection and preparation of suitable prodrug derivatives are described, for example, in Design of Prodrugs, ed. H. Bundgaard, Elsevier, 1985, and can be achieved using methods well known to those skilled in the art. All such prodrugs are within the scope of the combinations, pharmaceutical compositions, methods and kits of this invention.

The chemist of ordinary skill in the art will also recognize that certain compounds within the scope of this invention can exist in zwitterionic form, i.e., that certain compounds contain an amine portion and a carboxylic acid portion, which, depending upon the pH of the solution, may exist as a free amine and a free carboxylic acid or as a zwitterion in which the amine is protonated to form an ammonium ion and the carboxylic acid is deprotonated to form a carboxylate ion. All such zwitterions are included in this invention.

The chemist of ordinary skill in the art will also recognize that the pharmaceutical combinations contemplated by the present invention can exist in different stereoisomers. Specific stereoisomers may exhibit an ability to treat mental disorders with a more favorable efficacy or safety profile. The present invention includes all possible stereoisomers and geometric isomers of the active ingredients of each pharmaceutical combination, and includes not only racemic compounds but also optical isomers as well. In situations where tautomers, i.e., that an equilibrium exists between two isomers which are in rapid equilibrium with each other are possible, the present invention is intended to include all tautomeric forms.

The combinations of the present invention can be administered in a standard manner such as orally, parenterally, transmucosally (e.g., sublingually or via buccal administration), topically, transdermally, rectally, via inhalation (e.g., nasal or deep lung inhalation). Parenteral administration includes, but is not limited to, intravenous, intraarterial, intraperitoneal, subcutaneous, intramuscular, intrathecal, and intraarticular, or via a high pressure technique, like Powderject.™

For buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), or wetting agents (for example, sodium lauryl sulfate). The tablets can be coated according to methods well known in the art.

Such preparations can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

Solubilized forms of aryl-heterocyclics such as zirpasidone can be used in the pharmaceutical formulation of the invention.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinylpyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The combinations of this invention can also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the patient's condition and requirements.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and, a second therapeutic agent in an immediate release form. Alternatively, the atypical antipsychotic can be in immediate release form and the second therapeutic agent can be in sustained or controlled release form.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different compounds of this invention can be co-administered simultaneously or sequentially in any order, or as a single pharmaceutical composition comprising, for example, ziprasidone and a dopamine agonist as described above.

Since the present invention has an aspect that relates to the treatment of the diseases/conditions described herein with a combination of active ingredients which can be administered separately, the invention also relates to combining separate pharmaceutical compositions in kit form. The kit includes two separate pharmaceutical compositions: a ziprasidone composition and a dopamine agonist composition. The kit includes a container for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ”etc. Other variations of memory aids will be readily apparent to the skilled practitioner. A “daily dose” can be a single tablet or capsule or several pills or capsules to be taken on a given day. Also, a daily dose of the ziprasidone can consist of one tablet or capsule while a daily dose of the dopamine agonist can consist of several tablets or capsules or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment of the present invention, the treatment of psychosis and movement disorders associated with Parkinson's disease or subcortical dementias in a patient by the method of the present invention can include administering a triple combination pharmaceutical composition comprising;

a) an amount of a first therapeutic agent, said first therapeutic agent being ziprasidone, a pharmaceutically acceptable ziprasidone salt, a ziprasidone prodrug, or a pharmaceutically acceptable salt of said prodrug;

b) an amount of a second therapeutic agent, said second therapeutic agent being a first dopamine agonist; and

c) an amount of a third therapeutic agent, said third therapeutic agent being a second dopamine agonist.

wherein the amount (a), (b), and (c) are together effective in treating said psychosis and movement disorder associated with Parkinson's disease or a subcortical dementia disorder.

It will be understood that while the use of a single atypical antipsychotic as a first component compound is preferred, combinations of two or more atypical antipsychotics may be used as a first component if necessary or desired. Similarly, while the use of a single dopamine agonist as a second component compound is preferred, combinations of two or more of these agents may be used as a second component if necessary or desired.

The atypical antipsychotic of the present invention is useful alone or in combination with a second antipsychotic agent, for example, an atypical antipsychotic such as ziprasidone mesylate, a typical antipsychotic such as haloperidol, or a dopamine system stabilizer antipsychotic such as aripiprazole. It is preferred that if a second antipsychotic agent is used that they both administered to the patient in synergistic effective amounts. It is preferred that the total amount ranges from about 0.0001 to about 1000 mg/kg per day, more preferably from about 0.01 to about 100 mg/kg per day, and most preferably from about 0.1 to about 60 mg/kg per day.

Pharmaceutical compositions of use in the present invention will comprise one or both active compound(s) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampules, auto-injector devices or suppositories; for oral, parenteral, intranasal, sublingual or rectal administration, or for administration by inhalation or insufflation. For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a pharmaceutically acceptable salt thereof.

When referring to these preformulation compositions as homogeneous, it is meant that the active ingredients is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from about 1 to about 300 mg, for example about 1, 2, 5, 10, 25, 50 or 100 mg of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

When administered in combination, either as a single or as separate pharmaceutical composition(s), the ziprasidone and the dopamine agonist are presented in a ratio which is consistent with the manifestation of the desired effect. In particular, the ratio by weight of ziprasidone to the dopamine agonist will suitably be between about 0.001 to 1 and about 1000 to 1, and especially between about 0.01 to 1 and about 100 to 1.

The pharmaceutical combinations can be administered on a regimen of up to 6 times per day, preferably 1 to 4 times per day, especially 2 times per day, and most especially once daily.

### EXAMPLE 1

An example of a pharmaceutical composition that could be prepared according to the present invention is one made by combining ziprasidone with a dopamine agonist which is either: (a) carbidopa/levodopa, (b) pergolide (c) ropinirole or (d) pramipexole dihydrochloride in a pharmaceutically acceptable carrier. The composition contains respective amounts of ziprasidone and carbidopa/levodopa, levodopa, pergolide, ropinirole, or pramipexole to deliver on a daily basis between about 20 mg to about 200 mg ziprasidone and between about (a) 25/100 (i.e., 25 mg carbidopa and 100 mg levodopa) to 100/400 mg (i.e., 100 mg carbidopa and 400 mg levodopa); or (b) 1 mg to 3 mg pergolide; or (c) 0.25 mg to 9 mg ropinirole or (d) 0.125 mg to 4.5 mg pramipexole. The composition could be administered to a patient for the treatment of psychosis associated with Parkinson's disease or subcortical dementias on a daily, twice daily, three times daily, or four times daily basis.

### EXAMPLE 2

| | Quantity | | Quantity | |
|---|---|---|---|---|
| Ingredients | per cap | | per batch | |
| Ziprasidone | 20 | mg | 20 | gm |
| Carbidopa/levodopa | 25/100 | mg | 25 gm/100 gm | |
| Methocel E3 | 190 | mg | 38 | gm |
| Lactose monohydrate | 190 | mg | 38 | gm |
| Aerosil | 10 | mg | 2 | gm |
| SLS | 10 | mg | 2 | gm |

| | | | |
|---|---|---|---|
| GI. Acetic acid | q.s. | 40 | ml |

| | | |
|---|---|---|
| Total weight | 540 | mg |

Dissolve ziprasidone in acetic acid. Dissolve carbidopa/levodopa in the ziprasidone and acetic acid solution. Pass lactose, methocel and aerosil through a #40 mesh screen and mix well. Granulate the powder blend with the drug solution using multiple granulation 5 technique (3-4 times). Dry granules at 50° C. Pass the dried granules through a #60 screen and lubricate with sodium lauryl sulfate (SLS). The powder could be filled into capsules.

### EXAMPLE 3

| Ingredients | Quantity/Tab | |
|---|---|---|
| Ziprasidone | 20 | mg |
| Pergolide mesylate | 3 | mg |
| Lactose | 155.5 | mg |
| Crosscarmellose sodium (Intra) | 19.5 | mg |
| Crosscarmellose sodium (Extra) | 19.5 | mg |
| PEG 3000 | 50 | mg |
| Aerosil | 6.5 | mg |
| Magnesium stearate | 13 | mg |
| Povidone | 33 | mg |
| Isopropyl alcohol | 0.1 | ml |
| Dimethyl sulfoxide | 0.005 | ml |
| Total tablet weight | 300 | mg |

1) Pass ziprasidone, lactose and crosscarmellose (Intra) through a #60 screen 10 and mix.

2) Heat dimethyl sulfoxide and pergolide to form a solution; add isopropyl alcohol and continue heating; add PEG 3000 and povidone to form a clear solution.

3) Blend the mass of step 1 with the solution of step 2; pass through a #20 screen and dry for 30 minutes at 450° C.

4) Pass through a #40 screen and dry again at 450° C.

5) Mix with crosscarmellose (Extra), aerosil and magnesium stearate.

6) Compress the granulation into a tablet.

### EXAMPLE 4

A suspension formulation could be prepared by heating water to 70° C. followed by adding methylparaben while stirring at about 200 rpm with an overhead stirrer. After the parabens are completely dissolved, the temperature is lowered to about 30° C. The following components are then added in order: xanthan gum, xylitol, anhydrous citric acid, trisodium citrate dihydrate, polysorbate 80, NaCI, ziprasidone hydrochloride, carbidopa/levodopa.

It should be understood that the invention is not limited to the particular embodiments described herein, but that various changes and modifications may be made without departing from the spirit and scope of this novel concept as defined by the following claims.

What is claimed:

1. A pharmaceutical composition comprising (i) an amount of a first therapeutic agent which is an atypical antipsychotic and, (ii) an amount of a second therapeutic agent which is a dopamine agonist, and a pharmaceutically acceptable diluent or carrier, wherein the amounts of (i) and (ii) are together effective in treating psychosis and movement disorders associated with Parkinson's disease or subcortical dementias.

2. The pharmaceutical composition according to claim 1 wherein the first therapeutic agent is selected from the group consisting of olanzapine, aripiprazole, clozapine, risperidone, sertindole, quetiapine, amisulpride, asenapine, ziprasidone pharmaceutically acceptable salts thereof, prodrugs thereof, and pharmaceutically acceptable salts of said prodrugs.

3. The pharmaceutical composition of claim 1, wherein the first therapeutic agent is ziprasidone, a prodrug or a pharmaceutically acceptable salt thereof or a pharmaceutically acceptable salt of said prodrug.

4. The pharmaceutical composition of claim 1, wherein the first therapeutic agent is ziprasidone, a prodrug or a pharmaceutically acceptable salt thereof or a pharmaceutically acceptable salt of said prodrug, and the second therapeutic agent is levodopa/carbidopa, prodrugs or pharmaceutically acceptable salts thereof or pharmaceutically acceptable salts of said prodrug.

5. The pharmaceutical composition of claim 1, wherein the first therapeutic agent is ziprasidone, a prodrug or a pharmaceutically acceptable salt thereof or a pharmaceutically acceptable salt of said prodrug, and the second agent is pramipexole, a prodrug or a pharmaceutically acceptable salt thereof or a pharmaceutically acceptable salt of said prodrug.

6. A method for treating psychosis and a movement disorder in a subject afflicted with Parkinson's disease or a subcortical dementia comprising administering to said subject
a) an amount of a first therapeutic agent which is an atypical antipsychotic; and
b) an amount of a second therapeutic agent which is a dopamine agonist;
wherein the amounts of (a) and (b) are together effective in treating said psychosis and said movement disorder.

7. A method for treating a psychosis in a subject afflicted with Parkinson's disease comprising administering to said subject
a) an amount of a first therapeutic agent, which is an atypical antipsychotic; and
b) an amount of a second therapeutic agent, which is a dopamine agonist,
wherein the amounts of (a) and (b) are together effective in treating said psychosis associated with Parkinson's disease.

8. The method of claim 7, wherein (a) and (b) are administered to the subject in a single pharmaceutical composition.

9. The method of claim 7, wherein said dopamine agonist is levodopa, bromocriptine, carbidopa, pramipexole, pergolide mesylate, ropinirole, carbidopa/levodopa, a pharmaceutically acceptable salt or a prodrug thereof or a pharmaceutically acceptable salt of said prodrug.

10. The method of claim 9, wherein said dopamine agonist is carbidopa/levodopa, or a pharmaceutically acceptable salt thereof, or a prodrug thereof or a pharmaceutically acceptable salt of said prodrug.

11. The method of claim 10, wherein said dopamine agonist is pramipexole, or a pharmaceutically acceptable salt thereof, or a prodrug thereof or a pharmaceutically acceptable salt of said prodrug.

12. The method of claim 7, wherein the atypical antipsychotic is ziprasidone, a pharmaceutically acceptable salt thereof, a ziprasidone prodrug or ziprasidone prodrug pharmaceutically acceptable salt.

13. The method of claim 12, wherein the ziprasidone, ziprasidone salt, ziprasidone prodrug or ziprasidone prodrug salt is administered at a dosage of between about 5 mg to about 460 mg daily, preferably between about 10 mg to about 200 mg daily.

14. The method of claim 13, wherein the ziprasidone, ziprasidone salt, ziprasidone prodrug or ziprasidone prodrug salt is administered at a dosage of between about 20 mg to about 160 mg daily.

15. A method for treating a subcortical dementia disorder in a subject comprising administering to said subject
a) an amount of a first therapeutic agent, which is an typical antipsychotic; and
b) an amount of a second therapeutic agent, which is a dopamine agonist,
wherein the amounts of (a) and (b) are together effective in treating said subcortical dementia.

16. The method of claim 15, wherein (a) and (b) are administered to the subject in a single pharmaceutical composition additionally comprising a pharmaceutically acceptable vehicle, carrier or diluent.

17. The method of claim 15, wherein said therapeutic agents are administered by a method selected from the group consisting of oral, transmucosal, transdermal, nasal, pulmonary, buccal, parenteral, rectal, and sublingual.

18. The method of claim 17, wherein parenteral administration to the subject is selected from the group consisting of intravenous, intramuscular, subcutaneous, and intradermal.

19. The method of claim 15, wherein said dopamine agonist is selected from the group consisting of levodopa, bromocriptine, carbidopa, pramipexole, pergolide mesylate, ropinirole, carbidopa/levodopa, a pharmaceutically acceptable salt or a prodrug thereof or a pharmaceutically acceptable salt of said prodrug.

20. The method of claim 19, wherein said dopamine agonist is carbidopa/levodopa, or a pharmaceutically acceptable salt thereof, or a prodrug thereof or a pharmaceutically acceptable salt of said prodrug.

21. The method of claim 20, wherein said dopamine agonist is prampexole, or a pharmaceutically acceptable salt thereof, or a prodrug thereof or a pharmaceutically acceptable salt of said prodrug.

22. The method of claim 15, wherein said subcortical dementia is a dementia affecting a part of the brain beneath the cortex, a mixed cortical-subcortical dementia, or a subcortical dementia due to other general medical condition.

23. The method of claim 15, wherein the atypical antipsychotic is ziprasidone, a pharmaceutically acceptable salt of ziprasidone an ziprasidone prodrug or a ziprasidone prodrug pharmaceutically acceptable salt.

24. The method of claim 23, wherein the ziprasidone, ziprasidone salt, ziprasidone prodrug or ziprasidone prodrug salt is administered at a dosage of between about 5 mg to about 460 mg daily, preferably between about 1 Omg to about 200 mg daily.

25. The method of claim 24, wherein the ziprasidone, ziprasidone salt, ziprasidone prodrug or ziprasidone prodrug salt is administered at a dosage of between about 20 mg to about 160 mg daily.

26. The method of claim 25, wherein the ziprasidone, ziprasidone salt, ziprasidone prodrug or ziprasidone prodrug salt is administered at a dosage of between about 20 mg to about 80 mg daily.

27. A method for treating a psychosis associated with Parkinson's disease or a subcortical dementia disorder in a subject in need thereof comprising administering to said subject
a) an amount of a first therapeutic agent which is an atypical antipsychotic; and
b) an amount of a second therapeutic agent which comprises at least one dopamine agonist;
wherein the amounts (a) and (b) are together effective in treating said psychosis associated with Parkinson's disease or subcortical dementia disorder.

28. The method of claim 27, wherein said dopamine agonist is levodopa, bromocriptine, carbidopa, pramipexole, pergolide mesylate, ropinirole, carbidopa/levodopa, a pharmaceutically acceptable salt thereof, a prodrug thereof, or a pharmaceutically acceptable salt of said prodrug.

29. The pharmaceutical composition of claim 1, wherein the first and second therapeutic compounds are administered to the subject by a method selected from the group consisting of oral, intravenous, transmucosal, nasal, vaginal, pulmonary, transdermal, ocular, buccal, sublingual, intraperitoneal, intrathecal, intramuscular, rectal, or long term depot preparation.
